(19) United States
(12) Patent Application Publication
(10) Pub. No.: US 2022/0375618 A1
(43) Pub. Date: Nov. 24, 2022

(54) METHOD AND APPARATUS OF CALCULATING COMPREHENSIVE DISEASE INDEX

(71) Applicant: ELECTRONICS AND TELECOMMUNICATIONS RESEARCH INSTITUTE, Daejeon (KR)

(72) Inventors: Jae Hak YU, Daejeon (KR); Soon Hyun KOWN, Daejeon (KR); Se Jin PARK, Daejeon (KR); Jong Arm JUN, Daejeon (KR); Cheol Sig PYO, Daejeon (KR)

(21) Appl. No.: 17/741,151

(22) Filed: May 10, 2022

(30) Foreign Application Priority Data
May 11, 2021 (KR) 10-2021-0061001
Mar. 30, 2022 (KR) 10-2022-0039673

(51) Int. Cl. G16H 50/30; G16H 50/20; G16H 10/60

(52) U.S. Cl. CPC G16H 50/30; G16H 50/20; G16H 10/60

(57) ABSTRACT

A method of calculating a comprehensive disease index (CDI) is disclosed. The method includes analyzing pieces of medical data to calculate a disease risk value, analyzing pieces of vital data and vital data mapped to standard clinic guideline data among the pieces of vital data to calculate a disease severity value, and analyzing the disease risk value, the disease severity value, and medical knowledge information obtained from a medical knowledge base to calculate the CDI.

### CROSS-REFERENCE TO RELATED APPLICATIONS

This application claims the benefit of the Korean Patent Application Nos. 10-2021-0061001 filed on May 11, 2021, and 10-2022-0039673 filed on Mar. 30, 2022, which is hereby incorporated by reference as if fully set forth herein.

### BACKGROUND

### Field of the Invention

The present invention relates to a method and apparatus of calculating comprehensive disease index representing a disease risk level.

### Discussion of the Related Art

Recently, a healthcare service provides a service which predicts a disease risk level or a possibility of pathogenesis on the basis of medical examination data, electronic medical record (EMR) data, and personal health record (PHR) data.

The medical examination data, the EMR, and the PHR are not sufficient for a medical/clinical basis for determining a disease or a risk level (risk level value) of the disease. Therefore, it is required to develop technology for comprehensively analyzing a risk level of disease and/or an incidence probability (incidence possibility) of disease by using EMR/PHR data based on data associated with a standard clinic guideline or a disease screening tool and data usable as a medical/clinical basis such as a vital signal measured by a healthcare device.

### SUMMARY

An aspect of the present invention is directed to providing a method and apparatus of calculating comprehensive disease index representing a disease risk level by using EMR/PHR data based on data associated with a standard medical treatment guideline or a disease screening tool and data usable as a medical/clinical basis such as a vital signal measured by a healthcare device.

To achieve these and other advantages and in accordance with the purpose of the invention, as embodied and broadly described herein, there is provided a method of calculating a comprehensive disease index (CDI) by using a processor included in a computing device, the method including: analyzing pieces of medical data to calculate a disease risk value; analyzing pieces of vital data and vital data mapped to standard clinic guideline data among the pieces of vital data to calculate a disease severity value; and analyzing the disease risk value, the disease severity value, and medical knowledge information obtained from a medical knowledge base to calculate the CDI.

In an embodiment, the calculating of the disease risk value may include analyzing the medical data on the basis of a logistic regression analysis technique to calculate the disease risk value.

In an embodiment, the medical data may include medical examination data, electronic medical record data, and personal health record data.

In an embodiment, the calculating of the disease severity value may include: analyzing the vital data on the basis of a machine learning model and a deep learning model to calculate a prediction probability value representing a possibility of the disease; and analyzing the prediction probability value and the vital data mapped to the standard clinic guideline data to calculate the disease severity value.

In an embodiment, the calculating of the disease severity value may include: analyzing the vital data on the basis of a machine learning model and a deep learning model to calculate a prediction probability value representing a possibility of the disease; converting the vital data, mapped to the standard clinic guideline data, into a scale value representing a disease severity value on the basis of a rating scale defined in a standard clinic guideline item; and summating the prediction probability value and the scale vale to calculate the disease severity value.

In an embodiment, the vital data mapped to the standard clinic guideline data may include data associated with eye tracker mapped to a standard clinic guideline item including best gaze and visual field, gyro data and electromyogram (EMG) data mapped to a standard clinic guideline item including upper extremity exercise, lower extremity exercise, and limb ataxia, and voice recognition data mapped to a standard clinic guideline item including language aphasia and dysarthria.

In an embodiment, the calculating of the disease severity value may include mapping the standard clinic guideline data to the vital data on the basis of a mapping function which sets a mapping relationship between a standard clinic guideline item and the vital data.

In an embodiment, the calculating of the CDI may include analyzing a correlation between the disease risk value, the disease severity value, and the medical knowledge information on the basis of Bayesian theory to calculate the CDI.

In an embodiment, the calculating of the CDI may include: calculating a posterior probability of the standard clinic guideline data when the disease risk value, the disease severity value, and the medical knowledge information are given, on the basis of Bayesian theory; and calculating the calculated posterior probability as the CDI.

In another aspect of the present invention, there is provided an apparatus for calculating a comprehensive disease index (CDI), the apparatus including: a disease risk level calculation module configured to analyze pieces of medical data to calculate a disease risk value; a disease incidence prediction module configured to analyze pieces of vital data to calculate a prediction probability value representing a possibility of the disease; a disease severity calculation module configured to analyze vital data mapped to standard clinic guideline data among the pieces of vital data and the prediction probability value to calculate a disease severity value; and a CDI calculation module configured to analyze the disease risk value, the disease severity value, and medical knowledge information obtained from a medical knowledge base to calculate a CDI.

In an embodiment, the disease risk level calculation module may analyze the medical data on the basis of a logistic regression analysis technique to calculate a disease risk factor and the disease risk value corresponding to the disease risk factor.

In an embodiment, the disease incidence prediction module may analyze each of the pieces of vital data on the basis of a machine learning model and a deep learning model to calculate a prediction probability value representing a possibility of the disease.

In an embodiment, the disease severity calculation module may include: a data combiner configured to combine the standard clinic guideline data with the vital data; a weight calculator configured to calculate a weight corresponding to the prediction probability value and a scale value converted from the vital data mapped to the standard clinic guideline data; and an adder configured to summate the scale value, to which the weight is applied, and the prediction probability value, to which the weight is applied, to calculate the disease severity value.

In an embodiment, the data combiner may combine the standard clinic guideline data with the vital data on the basis of a mapping function which sets a mapping relationship between a standard clinic guideline item and the vital data.

In an embodiment, the CDI calculation module may calculate a posterior probability of the standard clinic guideline data when the disease risk value, the disease severity value, and the medical knowledge information are given, on the basis of a Bayesian learning model and calculates the calculated posterior probability as the CDI.

It is to be understood that both the foregoing general description and the following detailed description of the present invention are exemplary and explanatory and are intended to provide further explanation of the invention as claimed.

### BRIEF DESCRIPTION OF THE DRAWINGS

FIG. 1 is a block diagram of a computing device for implementing a method of calculating a comprehensive disease index (CDI) according to an embodiment of the present invention.

FIG. 2 is a schematic block diagram of an internal configuration of a disease risk level calculation module illustrated in FIG. 1.

FIG. 3 is a schematic block diagram of an internal configuration of a disease incidence prediction module illustrated in FIG. 1.

FIG. 4 is a detailed block diagram of a machine learning-based preprocessor and a machine learning-based disease incidence prediction model illustrated in FIG. 3.

FIG. 5 is a detailed block diagram of a deep learning-based disease incidence prediction model illustrated in FIG. 3.

FIG. 6 is a detailed block diagram of a disease severity calculation model illustrated in FIG. 1.

FIG. 7 is a diagram for describing a CDI calculation module illustrated in FIG. 1.

FIG. 8 is a flowchart illustrating a method of calculating a CDI according to an embodiment of the present invention.

FIG. 9 is a block diagram of a computing device for implementing a method of calculating a CDI illustrated in FIG. 8.

### DETAILED DESCRIPTION OF THE INVENTION

In the following description, the technical terms are used only for explain a specific exemplary embodiment while not limiting the present invention. The terms of a singular form may include plural forms unless referred to the contrary. The meaning of ‘comprise’, ‘include’, or ‘have’ specifies a property, a region, a fixed number, a step, a process, an element and/or a component but does not exclude other properties, regions, fixed numbers, steps, processes, elements and/or components.

Hereinafter, example embodiments of the invention will be described in detail with reference to the accompanying drawings. In describing the invention, to facilitate the entire understanding of the invention, like numbers refer to like elements throughout the description of the figures, and a repetitive description on the same element is not provided.

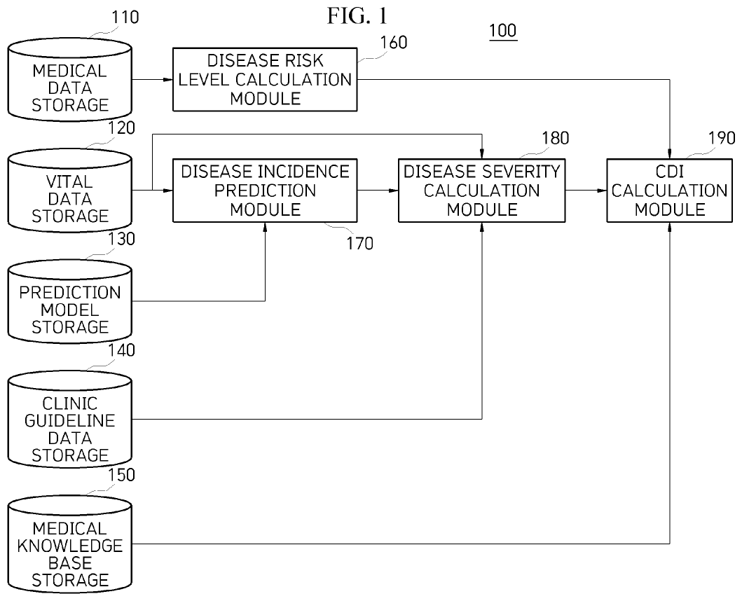

FIG. 1 is a block diagram of an apparatus 100 for implementing a method of calculating a comprehensive disease index (CDI) according to an embodiment of the present invention.

Referring to FIG. 1, the apparatus 100 for implementing a method of calculating a CDI according to an embodiment of the present invention may include a plurality of storages 110 to 150 and a plurality of modules 160 to 190 which are divided by processing units for calculating a CDI.

The plurality of storages 110 to 150 may each be a non-volatile storage medium or a computing device including the non-volatile storage medium. In FIG. 1, it is described that the plurality of storages 110 to 150 are disposed in the apparatus 100, but the plurality of storages 110 to 150 may be disposed outside the apparatus 100. In a case where the plurality of storages 110 to 150 are disposed outside the apparatus 100, the apparatus 100 may exchange various information with the plurality of storages 110 to 150 over a wired or wireless communication network (not shown).

In the present embodiment, five storages 110 to 150 are described, but some storages may be integrated into one storage or one storage may be subdivided into two or more storages on the basis of a detailed attribute of stored information.

To provide a detailed description on each storage, a medical data storage 110 may store medical data such as electronic medical record (EMR) data and personal health record (PHR) data. The medical data may be structuralized in a database form and may be stored in the medical data storage 110. Therefore, the medical data may be managed through a function of managing and controlling a database. The database may be a database management system (DBMS) and a relational database (RDB). Also, the medical data may include structured data and unstructured data such as a video and an image such as a letter string, a text, computed tomography (CT), and magnetic resonance imaging (MRI), and thus, may be implemented as a database such as appropriate not only SQL (NoSQL). The NoSQL may be implemented as document-based MongoDB, CouchDB, key value-based Redis, Bigtable-based Hadoop database (HBase), or Cassandra, but is not limited thereto.

The medical data storage 110 may provide appropriate medical data to the disease risk level calculation module 160 in response to a request of the disease risk level calculation module 160 described below.

A vital data storage 120 may store vital data including a vital signal such as electrocardiogram (ECG), electroencephalogram (EEG), electromyogram (EMG), or electrooculogram (EOG). The vital data may be structuralized in a database form and may be stored in the vital data storage 120. The vital data storage 120 may provide appropriate vital data to the disease incidence prediction module 170 in response to a request of the disease incidence prediction module 170 described below.

A prediction model storage 130 may store a prediction model such as a machine learning (ML) model and a deep learning (DL) model which have been learned previously. The prediction model storage 130 may provide an appropriate prediction model to the disease incidence prediction model 170 in response to a request of the disease incidence prediction model 170.

A clinic guideline data storage 140 may store data (hereinafter referred to as standard clinic guideline data) associated with a standard clinic guideline (or a critical pathway (CP)) or a disease screening tool. The standard clinic guideline data may be structuralized in a database form and may be stored in the clinic guideline data storage 140. The standard clinic guideline data may be a scale/score representing a physical disorder of a user or a patient occurring due to a specific disease. For example, when an application target of the present invention is stroke disease prediction, the standard clinic guideline data may include national institute of health stroke scale (NIHSS) data, face-arm-speech-time (FAST) data, and/or Cincinnati Prehospital stroke scale: CPSS) data. The clinic guideline data storage 140 may provide appropriate clinic guideline data to the disease severity calculation module 180 in response to a request of the disease severity calculation module 180 described below.

A medical knowledge base storage 150 may store a knowledge base associated with a medical domain. The medical knowledge base storage 150 may provide appropriate medical knowledge data to the CDI calculation module 190 in response to a request of the CDI calculation module 190 described below.

Each of the plurality of modules 160 to 190 may be a processor, including at least one central processing unit (CPU) and/or at least one graphics processing unit (GPU), or a computing device including the processor. Also, the plurality of modules 160 to 190 may each be a software module executed by at least one processor.

The disease risk level calculation module 160 may analyze and/or infer previous medical data (for example, EMR and PHR) provided from the medical data storage 110 to calculate a disease incidence risk factor and a disease incidence risk value.

The disease incidence prediction module 170 may a prediction probability value representing a possibility of disease by using the vital data provided from the vital data storage 120 and a machine learning model and/or a deep learning model provided from the prediction model storage 130.

The disease severity calculation module 180 may analyze and/or infer the prediction probability value provided from the disease incidence prediction module 170 and the standard clinic guideline data provided from the clinic guideline data storage 140 to calculate a disease severity value.

The CDI calculation module 190 may analyze and/or infer the disease risk factor and the disease risk value provided from the disease risk level calculation module 160, the disease severity value provided from the disease severity calculation module 180, and the medical knowledge provided from the medical knowledge base storage 150 to calculate a CDI.

As described above, a CDI representing a disease risk level may be calculated by using EMR/PHR data based on data associated with a standard clinic guideline or a disease screening tool and data usable as a medical/clinical basis such as a vital signal measured by a healthcare device, and thus, a disease of a user or a patient may be scientifically and objectively predicted and an optimal medical treatment may be provided based on a result of the prediction.

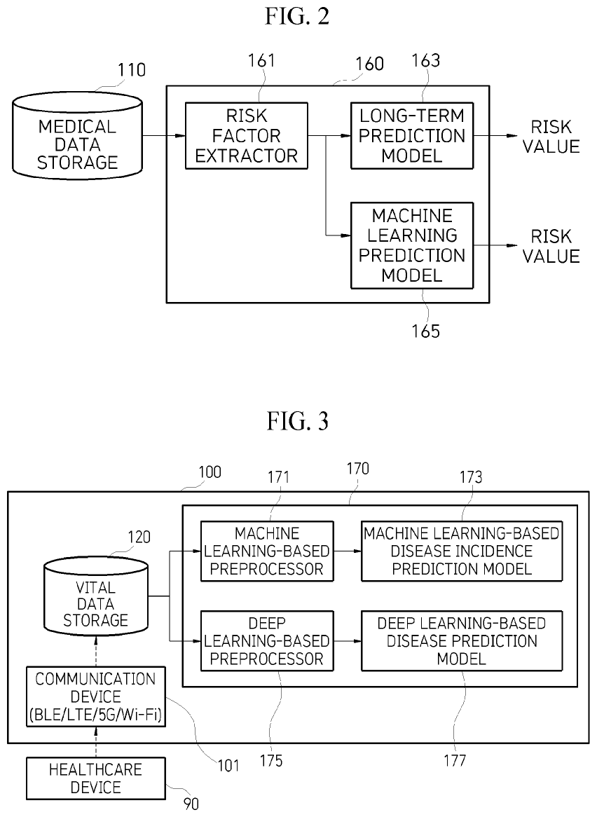

FIG. 2 is a schematic block diagram of an internal configuration of the disease risk level calculation module illustrated in FIG. 1.

Referring to FIG. 2, the disease risk level calculation module 160 according to an embodiment of the present invention may include a preprocessor 161, a long-term prediction model 163, and a machine learning prediction model 165.

The preprocessor 161 may preprocess the medical data (for example, EMR data and PHR data) provided from the medical data storage 110 to define risk factors and may extract the defined risk factors (significant parameters).

According to an embodiment of the present invention, the risk factors may include non-modifiable risk factors, modifiable risk factors, and other risk factors. Here, the modifiable risk factors may include risk factors having a medical/clinical basis and risk factors having an uncertain medical/clinical basis.

In stroke diseases, the non-modifiable risk factors may include age, gender, inherited factors, and low birthweight, the modifiable risk factors may include high blood pressure, diabetes or not, smoking or not, obesity, atrial fibrillation, dyslipidemia or not, and asymptomatic carotid stenosis, and the risk factors having an uncertain medical/clinical basis among the modifiable risk factors may include drinking, inflammation and infection, migraine, hypercoagulable state, and obstructive sleep apnea syndrome. Also, the other risk factors may include stress, underlying disease, drug, insufficient exercise, and accident record.

The long-term prediction model 163 may analyze the risk factors (the significant parameters) which are extracted by a risk factor extractor until a current time from a specific time, and thus, may predict and calculate a disease risk value (for example, a risk value of disease incidence after five or ten years) representing a disease possibility at a future time t. To this end, the long-term prediction model 163 may be implemented as a logistic regression analysis-based model, and for example, may be implemented as a cox proportional hazards model or a Weibull model.

The machine learning prediction model 165 may analyze risk factors (significant parameters) which are collected by the preprocessor 161 during a previous certain period, and thus, may predict and calculate a disease risk value at a current time. To this end, the machine learning prediction model 165 may be implemented as a model having a black/white box form, and for example, may be a decision tree model, a support vector machine (SVM) model, an artificial neural network (ANN) model, a Bayes-based model, or a random forest model.

The following Table 1 may show a logistic regression analysis result (a medical data-based risk value) of a man on the basis of the risk factors, and significance of risk factors may increase in the order of LDL (LDL cholesterol level), CRTN (serum creatinine level), HGB (haemoglobin level), FBS (fasting blood sugar level), BP_DIA (diastolic blood pressure), SGOT (AST (SGOT) level), and BP_SYS (systolic blood pressure) corresponding to significant parameters.

Table 1 shows a regression analysis result based on medical examination data of a man.

−0.02414 * G1E_BMI[body mass index] + 0.0003412 * G1E_BP_SYS[systolic
blood pressure] + 0.001584 * G1E_BP_DIA[diastolic blood pressure] + 0.02939 *
G1E_HGB[haemoglobin level] + −0.0008302 * G1E_FBS[fasting blood sugar
level] + 0.006524 * G1E_LDL[LDL cholesterol level] + −0.2704 *
G1E_CRTN[serum creatinine level] + 0.002487 * G1E_SGOT[AST (SGOT)
level] + −0.127

The following Table 2 may show a logistic regression analysis result of a woman, and risk factors may include LDL (LDL cholesterol level), CRTN (serum creatinine level), HGB (haemoglobin level), FBS (fasting blood sugar level), HA_RT (hearing (right)), HA_LT (hearing (left)) SGOT (AST (SGOT) level), and BP_SYS (systolic blood pressure) corresponding to significant parameters.

Table 2 shows a regression result based on medical examination data of a woman.

0.0002814 * G1E_BP_SYS[systolic blood pressure] + 0.02227 *
G1E_HGB[haemoglobin level] + −0.001445 * G1E_FBSG1E_FBS[fasting blood
sugar level] + 0.005004 * G1E_LDL[LDL cholesterol level] + −0.2574 *
G1E_CRTN[serum creatinine level] + 0.001305 * G1E_SGOT [AST (SGOT)
level] + 0.04569 * [G1E_HA_LT=1][hearing (left)] + 0.07977 *
[G1E_HA_RT=1] [hearing (right)] + −0.4601

In the present invention, a decision tree having a white box form in a prediction model based on a machine learning method will be described. Medical data used herein may include sixteen continuous factors, such as height, weight, systolic/diastolic blood pressure, blood sugar, and body mass index (BMI), and five discrete factors such as smoking and a drinking exercise count.

When a confidence factor value corresponding to a setting value of the decision tree is set to 0.25 and the minimum number of nodes is set to 2, it may be accurately predicted that a normality/risk or not of diseases (stroke diseases) of 65 or more-year-old aged persons is 77.20%. Particularly, because ID3 which is a representative algorithm of the decision tree has a demerit where an attribute having a value of a large range is selected as an upper node, the present invention has used a C4.5 decision tree algorithm which is the most advanced and has classification and prediction performance already verified. An entropy and an amount of information of an attribute of each node configuring the decision tree may be expressed as the following Equation 1.

$$H ( Y ) = - {\sum }_{y ∈ Y} p ( y ) {log}_{2} ( p ( y ) ) . H ( Y \vert X ) = - {\sum }_{x ∈ 𝒳} p ( x ) {\sum }_{y ∈ Y} p ( y \vert x ) {log}_{2} ( p ( y \vert x ) ) \qquad [ Equation 1 ]$$

Therefore, an information gain may be defined as the following Equation 2.

Gain=H(Y)+H(X)−H(X,Y) [Equation 2]

The information gain may be normalized as expressed in the following Equation 3 by using split information defined similar to an entropy.

$$Split info ( Y ) = - {\sum }_{i = 1}^{n} \frac{\vert {Y}_{i} \vert}{\vert Y \vert} \times {log}_{2} ( P \frac{\vert {Y}_{i} \vert}{\vert Y \vert} ) \qquad [ Equation 3 ]$$

An attribute having a maximum gain ratio may be selected as a split attribute as expressed in the following Equation 4.

$$Gain ratio ( Y ) = \frac{gain ( Y )}{Split info ( Y )} \qquad [ Equation 4 ]$$

FIG. 3 is a schematic block diagram of an internal configuration of the disease incidence prediction module illustrated in FIG. 1.

Referring to FIG. 3, the disease incidence prediction module 170 according to an embodiment of the present invention may include a machine learning-based preprocessor 171 and a machine learning-based disease incidence prediction model 173, and moreover, may further include a deep learning-based preprocessor 175 and a deep learning-based disease prediction model 177.

First, a healthcare device 90 may measure vital data (for example, ECG data, EEG data, EMG data, EOG data, and MOTION data) based on a vital signal in real time and may transmit the vital data to a communication device 101 on the basis of a real-time streaming scheme by using wired/wireless communication. Here, the wireless communication may be, for example, BLE communication, Wi-Fi communication, LTE communication, or 5G communication.

The communication device 101 may store the vital data, transmitted from the healthcare device 90, in the vital data storage 120, and data stored in the vital data storage 120 may be preprocessed by the machine learning-based preprocessor 171 and may be additionally preprocessed by the deep learning-based preprocessor 175.

Preprocessing performed by the machine learning-based preprocessor 171 according to an embodiment of the present invention may include a process of extracting pieces of feature data corresponding to each vital data and a process of selecting pieces of significance data among the extracted feature data, and depending on the case, may include a normalization and regularization process performed on the selected significance data.

Preprocessing performed by the deep learning-based preprocessor 175 according to an embodiment of the present invention may include a process of parsing raw data corresponding to the vital data, a process of scaling a sampling rate of the raw data, and a process of compressing a length or a size of an input vector representing the raw data by using principal component analysis (PCA), independent component analysis (ICA), fast Fourier transform (FFT), and integral average value (IAV).

Based on a design, only one of two preprocessors 171 and 175 may operate, or all of the two preprocessors 171 and 175 may operate.

Moreover, the machine learning-based preprocessor 171 may be executed in a single mode for one learning and prediction model, or may be executed in a multimode so as to provide a service which is set to a multimodal.

The machine learning-based disease incidence prediction model 173 may predict a possibility of disease in real time on the basis of data preprocessed by the machine learning-based preprocessor 171 and may calculate a prediction probability value representing a result of the prediction. To this end, the machine learning-based disease incidence prediction model 173 may be implemented as a machine learning model.

Likewise, the deep learning-based disease prediction model 177 may predict a possibility of disease in real time on the basis of data preprocessed by the deep learning-based preprocessor 175 and may calculate a prediction probability value representing a result of the prediction. To this end, the deep learning-based disease prediction model 177 may be implemented as a deep learning model.

The machine learning-based disease incidence prediction model 173 and the deep learning-based disease prediction model 177 may be progressively updated through self-learning, and updated models may be stored in the prediction model storage 130 again. In this case, although not shown in FIG. 3, a verifier may be connected to output terminals of the updated prediction models 173 and 177, a medical staff or an expert may verify the accuracy of the prediction models 173 and 177 by using the verifier, and the prediction model storage 130 may store only prediction models 173 and 177, verified to have high accuracy, of the updated prediction models 173 and 177.

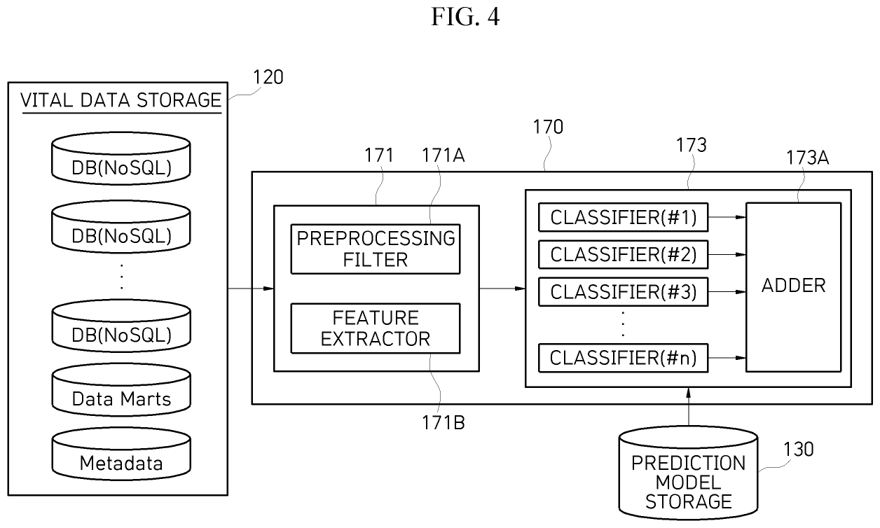

FIG. 4 is a detailed block diagram of the machine learning-based preprocessor and the machine learning-based disease incidence prediction model illustrated in FIG. 3.

Referring to FIG. 4, the vital data storage 120 may store vital data on the basis of a scheme such as NoSQL-based distribution storage or data mart, but is not limited thereto.

The machine learning-based preprocessor 171 may include a preprocessing filter 171A and a feature extractor 171B. The preprocessor 171A may filter missing value data or an error for each vital data, and the feature extractor 171B may extract a predefined significant feature having a medical/clinical meaning from the filtered vital data in real time.

To this end, the feature extractor 171B may include fast Fourier transform (FFT), wavelet transform: (WT), principal component analysis (PCA), and independent component analysis (ICA).

According to an embodiment, in a case where the preprocessor 171 performs preprocessing on ECG data, the preprocessor 171 may extract feature data, such as RRI-segment (segment between R-peaks in an ECG signal), QRA-segment (segment consisting of Q wave, R wave, and S wave in the ECG signal), and ST-segment (segment between an end point of S wave and a time of T wave in the ECG signal), from the ECG data.

Moreover, the preprocessor 171 may select and reduce pieces of significant feature data from among the extracted feature data on the basis of correlation feature selection and/or cross-correlation coefficient technique.

Vital signals may have a time-series characteristic, and it may be important that a decision function is defined by simultaneously inputting two or more multi vital signals, instead of a single vital signal, to a prediction model so as to predict a disease in a service (for example, walking, driving, and sleeping).

A cross-correlation coefficient of a time-series vital signal may be implemented by the following Equations. First, when it is assumed that n pieces of time-series data are two vital signal data (for example, ECG data and EMG data), ECG may be defined as x=x₁, x₂, . . . , xₙ and EMG may be defined as y=y₁, y₂, . . . , yₙ, on the basis of the following Equation 5.

$${C}_{xy} = \{ \begin{matrix}\frac{1}{n} {\sum }_{t = 1}^{n - k} ( {x}_{t} - μ ( x ) ) ( {y}_{t + k} - μ ( y ) ) , k = 0 , 1 , … , n - 1 \\ \frac{1}{n} {\sum }_{t = 1 - k}^{n} ( {x}_{t} - μ ( x ) ) ( {y}_{t + k} - μ ( y ) ) , k = - 1 , … , - n + 1\end{matrix} \qquad [ Equation 5 ]$$

A sample cross-correlation coefficient may be induced as expressed in the following Equation 6. Here, r_{xy}^{(k)} may have a value between −1 and +1 on the basis of Equation 5.

$${r}_{xy} ( k ) = \frac{{C}_{xy} ( k )}{\sqrt{{C}_{xx} ( 0 ) {C}_{yy} ( 0 )}} \qquad [ Equation 6 ]$$

Here, it may be unable to calculate a sample cross-correlation coefficient corresponding to a total period of the n pieces of time-series data, and thus, the n pieces of time-series data may be decomposed based on a size equal to m so as to optimally extract a vital signal-based feature and requirement of a system. The n pieces of time-series data may be decomposed based on a smaller size, and thus, a memory and a storage of a device may be efficiently used. However, when a sample size is defined to be very short, it may be unable to extract significance features (for example, RRI-segment, QRS-segment, and ST-segment of ECG) of a vital signal).

Therefore, in an embodiment of the present invention, in ECG, a minimum decomposition time may be set to 6 sec. When 6 sec which is a decomposition time of ECG is defined as p, n=pm may be established. A method of setting a decomposition time to p may be described for example, and requirement of a service or each vital signal may be decomposed and extracted as various values. Accordingly, an interval cross-correlation coefficient of time-series data such as a vital signal may be induced as expressed in the following Equation 7. Here, when an arbitrary interval is j∈[1, 2, . . . , p], a time-series vital signal ECG may be represented as x(j)=x₁^{j}, x₂^{j}, . . . , xₘ^{j} and EMG may be represented as y(j)=y₁^{j}, y₂^{j}, . . . , yₘ^{j}.

$${r}_{xy}^{j} ( k ) = \frac{{c}_{xy}^{j} ( k )}{\sqrt{{c}_{xx}^{j} ( 0 ) {c}_{yy}^{j} ( 0 )}} \qquad [ Equation 7 ]$$

Extracted and compressed significant features may solve a problem dependent on a measurement unit of data through a normalization and regularization process.

When one feature is expressed as a value of a relatively small unit in more detail, a relative value of a feature may have a large range, and thus, all vector values may be set within a range of −1 to 1 or 0.0 to 1.0 for each feature. However, the present invention is not limited thereto, and a representative regularization technique may include a minimum-maximum method, a Z-score method, and a decimal-scaling method.

The disease incidence prediction module 170 may read the machine learning model stored in the prediction model storage 130 and may load the machine learning model into a memory (not shown), and thus, may complete a process of preparing for execution of the machine learning-based disease incidence prediction model 173.

The machine learning-based disease incidence prediction model 173 may include n number of classifiers #1 to #n and an adder 173A loaded from the prediction model storage 130, so as to calculate a prediction probability value representing a possibility of disease on the basis of vital data preprocessed by the machine learning-based preprocessor 171.

According to an embodiment of the present invention, the n pieces of data preprocessed by the preprocessor 171 may be input to the n classifiers #1 to #n on the basis of a one-to-one method. For example, one piece of preprocessed data may be input to one classifier, and the n classifiers #1 to #n may calculate different prediction probability values on the basis of different pieces of preprocessed data.

According to another embodiment of the present invention, the n pieces of data preprocessed by the preprocessor 171 may be input to the n classifiers #1 to #n on the basis of a one-to-n method. For example, one piece of preprocessed vital data may be simultaneously input to the n classifiers #1 to #n, and the n classifiers #1 to #n may calculate different prediction probability values on the basis of the one piece of preprocessed vital data. Subsequently, a process of summating the prediction probability values calculated by the n classifiers #1 to #n or calculating an average value of the prediction probability values may be further performed.

According to another embodiment of the present invention, the n pieces of data preprocessed by the preprocessor 171 may be input to one classifier on the basis of an n-to-one method. For example, the n pieces of preprocessed data may be defined as a single feature vector, and then, the single feature vector may be input to one classifier and the classifier may calculate a prediction probability value on the basis of the single feature vector.

According to another embodiment of the present invention, the n pieces of data preprocessed by the preprocessor 171 may be input to the n classifiers #1 to #n on the basis of an n-to-one method. For example, a prediction probability value may be calculated by using n pieces of processed vital data as an input of each classifier.

A weight θ divided for each service may be set to the n classifiers #1 to #n, the n classifiers #1 to #n where the weight θ is set may calculate prediction probability values, and the calculated prediction probability values may be summated by the adder 173A and may be calculated in a disease score form as expressed in the following Equation 8.

$$Disease {Score}_{i} = {\sum }_{n = 1}^{N} {θ}_{n} ( {x}_{n} ) \qquad [ Equation 8 ]$$

Here, θ may have a value between 0.0 and 1.0, and a sum thereof may be 1.0, and n may be a factor representing vital data or a classifier.

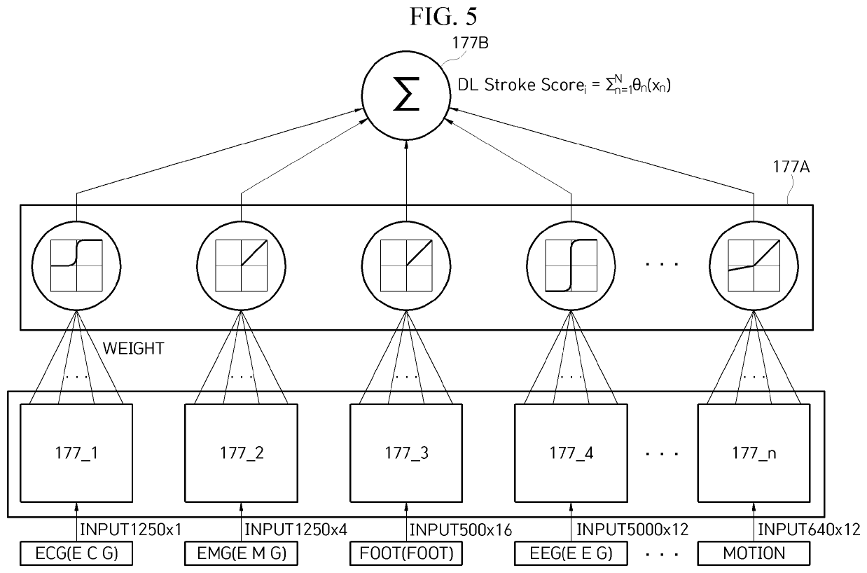

FIG. 5 is a detailed block diagram of the deep learning-based disease incidence prediction model illustrated in FIG. 3.

Referring to FIG. 5, disease prediction may be performed by using single vital data as a single deep learning model, but when a weight and a feature vector of each vital data are shared, a calculation time and an accuracy of prediction may be reduced.

A significance of vital data used for each service may be determined based on an interval cross-correlation coefficient in Equation 7, and finally, a probability value where a disease occurs may be calculated as a value of 0.0 to 1.0 in a softmax function.

In FIG. 5, an example is illustrated where a multi vital data including ECG data of 1 channel, EMG data of 4 channel, Foot data of 16 channel, EEG data of 12 channel, and motion data of 12 channel is used as an input vector.

The deep learning-based disease prediction model 177 may include n number of deep learning models 177_1 to 177_n divided for each vital data, n number of activation functions 177A, and an adder 177B.

Each deep learning model may be implemented as one of 1D-convolutional neural networks (CNN), long short-term memory (LSTM) of recurrent neural networks (RNN), and multi 1D-CNN.

The activation function may determine whether a total sum of output values of deep learning models obtained by multiplying weights causes activation. Each activation function may be one of a sigmoid function, a rectified linear unit (ReLU) function, a tanh function, and a leaky ReLU function.

As described above, the deep learning-based disease prediction model 177 may be designed as an optimal model where the deep learning models 177_1 to 177_n divided for each vital data are combined with the activation functions 177A.

Prediction probability values calculated by the deep learning models 177_1 to 177_n and the activation functions 177A may be summated by the adder 177B which is an upper layer. In this case, a weight θ may be assigned to each prediction probability value, and the adder 177B may summate prediction probability values to which the weight θ is assigned.

Based on an opinion of a medical expert, a weight may be set to about 1.0 in association with vital data where significance is high, or a weight may be set to about 0.0 in association with vital data where significance is low.

A final prediction probability value of a stroke disease calculated by the adder 177B may be expressed as the following Equation 9.

$$DL Stroke {Score}_{i} = {\sum }_{n = 1}^{N} {θ}_{n} ( {x}_{n} ) \qquad [ Equation 9 ]$$

Here, θₙ may denote a weight of n^{th} vital data, and xₙ may denote a prediction probability value based on the n^{th} vital data.

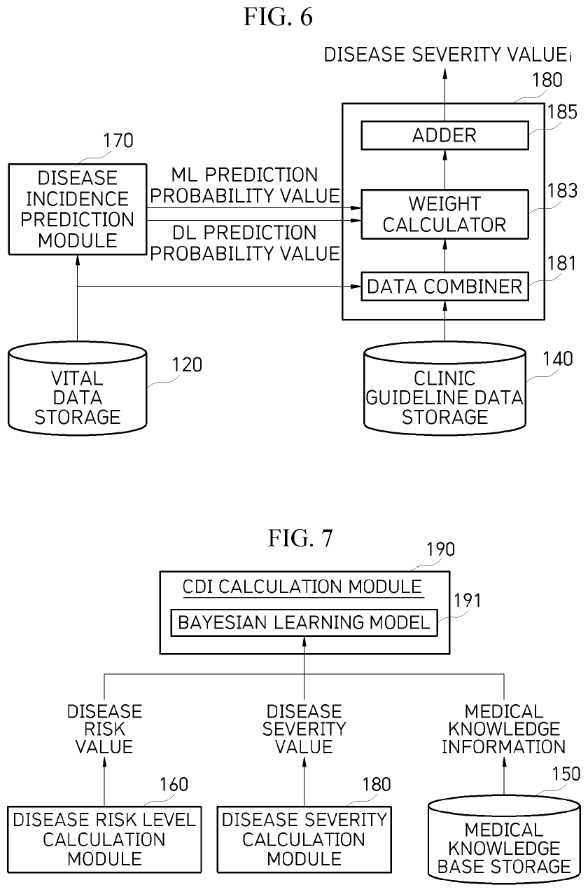

FIG. 6 is a detailed block diagram of the disease severity calculation model illustrated in FIG. 1.

Referring to FIG. 6, the disease severity calculation model 180 according to an embodiment of the present invention may include a data combiner 181, a weight calculator 183, and an adder 185.

The data combiner 181 may combine vital data, provided from the vital data storage 120, with standard clinic guideline data provided from the clinic guideline data storage 140. According to an embodiment of the present invention, the data combiner 181 may map vital data and clinic item data defined by the standard clinic guideline data by using a pre-defined mapping function or mapping table.

When NIHSS in the standard clinic guideline data is assumed, main clinic items of NIHSS associated with a stroke disease may include items for measuring level of consciousness, best gaze, visual field, facial palsy, upper extremity exercise, lower extremity exercise, limb ataxia, sensation, language aphasia, dysarthria, extinction and inattention, and distal movement.

The following Table 1 may show a mapping result between vital data and main clinic item data of NIHSS on the basis of a mapping function (a mapping table).

TABLE 3

| Main Clinic Items | Vital Data |
|---|---|
| Level of | None |
| Consciousness | |
| Best Gaze | Eye Tracker |
| Visual Field | Eye Tracker |
| Facial Palsy | None |
| Upper Extremity | EMG & Gyro |
| Exercise | |
| Lower Extremity | EMG & Gyro |
| Exercise | |
| Limb Ataxia | EMG & Gyro |
| Sensation | None |
| Language Aphasia | Voice Recognition |
| Dysarthria | Voice Recognition |
| Extinction and | None |
| Inattention | |
| Distal Movement | None |

As in Table 3, based on a mapping function, vital data such as EMG may be mapped (combined) to a clinic item such as upper extremity exercise, lower extremity exercise, and limb ataxia, vital data associated with eye tracker may be mapped to a clinic item such as best gaze and visual field, and vital data such as voice recognition may be mapped to a clinic item such as language aphasia and dysarthria.

The data combiner 181 may convert vital data, mapped to each clinic item, into a scale value representing a severity of a disease on the basis of a rating scale defined in each clinic item.

In order to calculate a severity of a disease, in an embodiment of the present invention, data obtained by combining real-time collected vital data with standard clinic guideline data which is a tool widely used in medical institutions may be used as data for calculating a severity of a disease.

An operation of predicting a severity (risk level) of a disease on the basis of vital data simply collected and measured in real time may be medically/clinically risk. Accordingly, the present invention may be characterized in that data where standard clinic guideline data is combined with vital data is used as information for calculating a severity of a disease.

The weight calculator 183 may calculate a weight (Weightθ 1) of a scale value converted from vital data mapped to standard clinic guideline data, and the weight may be determined based on a cross-correlation coefficient expressed as Equations 6 and 7 representing a correlation between the standard clinic guideline data and the vital data.

Moreover, the weight calculator 183 may calculate a weight (Weightθ 2) of a machine learning (ML)-based prediction probability value and/or a deep learning (DL)-based prediction probability value calculated by the disease incidence prediction module 170.

The adder 185 may summate the scale value, to which the weight (Weightθ 1) is applied, and the machine learning (ML)-based prediction probability value and/or deep learning (DL)-based prediction probability value, to which the weight (Weightθ 2) is applied, to finally generate a disease severity value.

The following Equation 10 may represent a weight of a machine learning/deep learning-based prediction probability value or a scale value converted from vital data on the basis of a scale defined in each item of the standard clinic guideline data, and the following Equation 11 may represent a disease severity value calculated as a machine learning/deep learning-based prediction probability value to which a weight is applied and a scale value to which a weight is applied.

$${Weight}_{θ} = {\sum }_{n = 1}^{N} {θ}_{n} < {f}_{{w}_{L} , {w}_{2}^{n} , {w}_{s}^{n} , … , {w}_{L - 1}^{m}}^{{n}_{n}} ( {x}_{i} ) , {W}_{L}^{n} > + b \qquad [ Equation 10 ]$$

$$Disease Severity {Value}_{i} = {\sum }_{i = 1}^{n} {θ}_{i} {Model}_{i} + {\sum }_{i = 1}^{n} {θ}_{i} {NIHSS}_{i} \qquad [ Equation 11 ]$$

FIG. 7 is a diagram for describing the CDI calculation module illustrated in FIG. 1.

Referring to FIG. 7, the CDI calculation module 190 may calculate a CDI on the basis of a risk factor and/or a risk value of a disease provided from the disease risk level calculation module 160, a disease severity value provided from the disease severity calculation module 180, and medical knowledge information provided from a medical knowledge base storage.

In order to calculate the CDI, the CDI calculation module 190 according to an embodiment of the present invention may calculate the CDI on the basis of a Bayesian learning model 191. The Bayesian learning model 191 may be implemented as a machine learning model or a deep learning model on the basis of Bayesian theory.

The Bayesian learning model 191 may calculate a posterior probability P(ωᵢ|x) as expressed in the following Equation 16 on the basis of a disease risk value based on medical data, a disease severity value, and medical knowledge information according to the Bayesian theory and may calculate the calculated posterior probability P(ωᵢ|x) as the CDI.

Hereinafter, a CDI calculation process based on the Bayesian theory will be described.

The disease risk value, the disease severity value, and the medical knowledge information used as an input of the Bayesian learning model 191 may fundamentally have a continuous value, and thus, may be defined as a continuous probability distribution based on a probability density function (PDF) as in the following Equation 12.

$$Continuous Probability Distribution \{ \begin{matrix}μ = {\int }_{- ∞}^{∞} xp ( x ) dx \\ {σ}^{2} = {\int }_{- ∞}^{∞} {( x - μ )}^{2} p ( x ) dx\end{matrix}$$

Because accuracy is reduced when only one feature is used for calculating or predicting a CDI in healthcare or medical field, in the present embodiment, a final CDI may be calculated based on the disease risk value, the disease severity value, and the medical knowledge information.

In order to apply all of the disease risk value, the disease severity value, and the medical knowledge information, several random parameters may consist of a random vector. Here, the random vector may be expressed as a d-dimensional vector x=x₁, x₂, x₃, . . . , x_{d})^{T}, and an average vector may be expressed as μ=(μ₁, μ₂, μ₃, . . . , μ_{d})^{T}. The average vector may be calculated as expressed in the following Equation 13, and R^{d} may denote a d-dimensional real number space.

$$μ = {\int }_{{R}^{d}} xp ( x ) dx \qquad [ Equation 13 ]$$

In order to apply all of the disease risk value, the disease severity value, and the medical knowledge information, a variance σᵢ² of an i^{th} element of a random vector may be needed, and a covariance σᵢⱼ between xᵢ and xⱼ having a significant statistical characteristic and meaning may be needed. The following Equation 14 may represent a covariance matrix Σ. Here, because σᵢⱼ=σⱼᵢ, Σ may be a symmetric matrix.

$$\sum = ( \begin{matrix} & {σ}_{11} & {σ}_{12} & … & {σ}_{1 d} & \\ & {σ}_{21} & {σ}_{22} & … & {σ}_{2 d} & \\ \cdot & & & \cdot & & \\ \cdot & & & & \cdot & \\ \cdot & & & & & \cdot \\ & {σ}_{d 1} & {σ}_{d 2} & … & {σ}_{dd} & \end{matrix} ) = ( \begin{matrix} & {σ}_{1}^{2} & {σ}_{12} & … & {σ}_{1 d} & \\ & {σ}_{21} & {σ}_{2}^{2} & … & {σ}_{2 d} & \\ \cdot & & & \cdot & & \\ \cdot & & & & \cdot & \\ \cdot & & & & & \cdot \\ & {σ}_{d 1} & {σ}_{d 2} & … & {σ}_{d}^{2} & \end{matrix} ) \qquad [ Equation 14 ]$$

A covariance of a disease risk value based on medical data, a disease severity value based on a vital signal, and medical knowledge information based on the medical data may be calculated as expressed in the following Equation 15. The covariance may express a relationship between random parameters constituting a random vector, and thus, may be a criterion for calculating significance or a correlation between the disease risk value based on the medical data, the disease severity value based on the vital signal, and the medical knowledge information.

$$\sum = {\int }_{{R}^{d}} ( x - μ ) {( x - μ )}^{T} p ( x ) dx \qquad [ Equation 15 ]$$

Based on the Bayesian theory, a final CDI may be calculated as a posterior probability P(ωᵢ|x) of the following Equation 16 from the disease risk value based on the medical data, the disease severity value, and the medical knowledge information.

$$P ( {ω}_{i} \vert x ) = \frac{P ( {ω}_{i} ) P ( x \vert {ω}_{i} )}{P ( x )} = \frac{P ( x \vert {ω}_{i} )}{P ( x )} \cdot P ( {ω}_{i} ) \qquad [ Equation 16 ]$$

Here, x may denote an input vector corresponding to information and/or a value input to the Bayesian learning model 191. Also, ωᵢ may be standard clinic guideline data (continuous probability value) and may classify a severity of a stroke disease as a risk level of NUNS No Stroke Symptoms, Minor Stroke, Moderate Stroke, Severe Stroke}, and ωᵢ may be finally calculated as a continuous value on the basis of the purpose of a system or a service. Also, P(ωᵢ) may be a prior probability of ωᵢ, P(x|ωᵢ) may be a likelihood probability of x when ωᵢ is given, and P(x) may be a normalizing constant. Also, P(ωᵢ|x) may be a posterior probability of ωᵢ when x is given.

In Equation 16, because a discrete CDI is calculated, it may be required to consider the calculation of a Bayesian-based CDI capable of extending to N number of classifications having a continuous value. In this case, a minimum error Bayesian classifier may be used.

In order to calculate a CDI on the basis of the minimum error Bayesian classifier, N number of posterior probabilities may be calculated, and then, when

$$k = arg {max}_{i} P ( x \vert {ω}_{i} ) P ( {ω}_{k} ) ,$$

x may be classified as ωₖ to have a largest posterior probability.

A minimum error Bayesian classification of N classifications may be finally obtained as in the following Equation 17, R including x among R₁, R₂, R₃, . . . , R_{N} may be determined for minimizing average loss D as in the following Equation 18. That is, when x is included in loss equal to qᵢ may occur, and thus, a decision rule for minimizing D may be expressed as the following Equation 18.

$$D = {\sum }_{i = 1}^{N} {\int }_{{R}_{i}} ( ( {\sum }_{j = 1}^{N} {c}_{j}^{i} P ( x \vert {ω}_{j} ) ) P ( {ω}_{j} ) ) dx \qquad [ Equation 17 ]$$

$$\begin{matrix}x is k = arg {min}_{i} {q}_{i} , \\ {q}_{i} = {\sum }_{j = 1}^{N} ( {\sum }_{j = 1}^{N} {c}_{j}^{i} P ( x \vert {ω}_{j} ) ) P ( {ω}_{j} )\end{matrix} \} \qquad [ Equation 18 ]$$

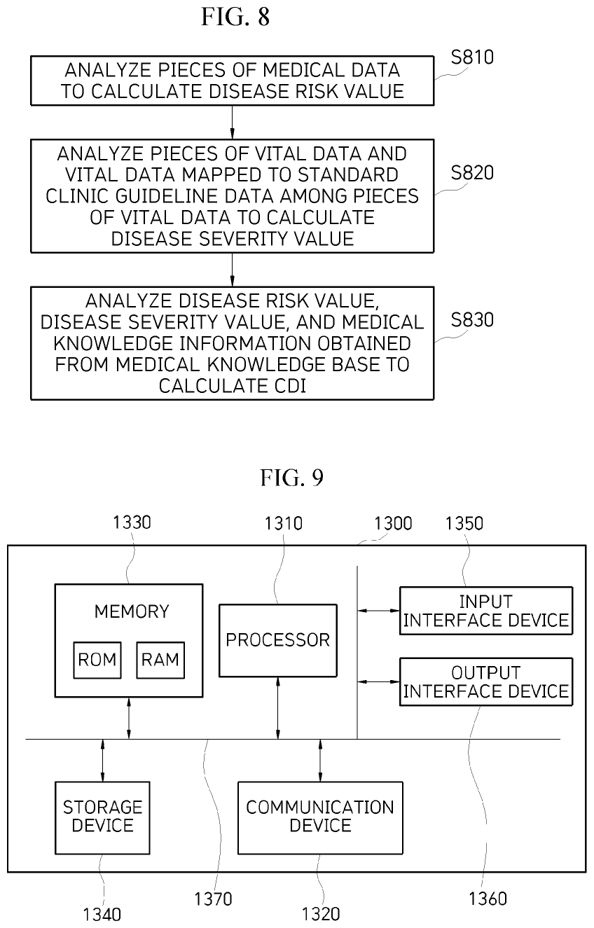

FIG. 8 is a flowchart illustrating a method of calculating a CDI according to an embodiment of the present invention.

Unless described, a main element for performing each step may be at least one processor (at least one CPU and/or at least one GPU) included in a computing device, or may be a hardware and/or software module executed and/or controlled by the at least one processor. Here, the hardware and/or software module may be a corresponding element among the elements 160, 170, 180, and 190 illustrated in FIG. 1.

Referring to FIG. 8, first, in step S810, a process of analyzing pieces of medical data to calculate a disease risk value may be performed by at least one processor or the disease risk level calculation module 160 executed and/or controlled by the at least one processor.

Subsequently, in step S820, a process of analyzing pieces of vital data and vital data mapped to standard clinic guideline data among the pieces of vital data to calculate a disease severity value may be performed by at least one processor or the disease severity calculation module 180 executed and/or controlled by the at least one processor.

Subsequently, in step S830, a process of analyzing the disease risk value, the disease severity value, and medical knowledge information obtained from a medical knowledge base to calculate the CDI may be performed by at least one processor or the CDI calculation module 190 executed and/or controlled by the at least one processor.

According to an embodiment of the present invention, S810 may be a step of analyzing the medical data on the basis of a logistic regression analysis technique to calculate the disease risk value.

According to an embodiment of the present invention, the medical data may include medical examination data, electronic medical record data, and personal health record data.

According to an embodiment of the present invention, S820 may include a process of analyzing the vital data on the basis of a machine learning model and a deep learning model to calculate a prediction probability value representing a possibility of disease and a process of analyzing the prediction probability value and vital data mapped to the standard clinic guideline data to calculate the disease severity value.

According to an embodiment of the present invention, S820 may include a process of analyzing the vital data on the basis of the machine learning model and the deep learning model to calculate a prediction probability value representing a possibility of disease, a process of converting the vital data, mapped to the standard clinic guideline data, into a scale value representing a disease severity value on the basis of a rating scale defined in a standard clinic guideline item, and a process of summating the prediction probability value and the scale vale to calculate the disease severity value.

According to an embodiment of the present invention, the vital data mapped to the standard clinic guideline data may include data associated with eye tracker mapped to a standard clinic guideline item including best gaze and visual field, gyro data and EMG data mapped to a standard clinic guideline item including upper extremity exercise, lower extremity exercise, and limb ataxia, and voice recognition data mapped to a standard clinic guideline item including language aphasia and dysarthria.

According to an embodiment of the present invention, S820 may include a process of mapping the standard clinic guideline data to the vital data on the basis of a mapping function which sets a mapping relationship between a standard clinic guideline item and the vital data.

According to an embodiment of the present invention, S830 may include a process of analyzing a correlation between the disease risk value, the disease severity value, and medical knowledge information on the basis of the Bayesian theory to calculate the CDI.

According to an embodiment of the present invention, S830 may include a process of calculating a posterior probability of the standard clinic guideline data when the disease risk value, the disease severity value, and medical knowledge information are given, on the basis of the Bayesian theory and a process of calculating the calculated posterior probability as the CDI.

FIG. 9 is a block diagram of a computing device 1300 for implementing a method of calculating a CDI illustrated in FIG. 8.

Referring to FIG. 9, the computing device 1300 may include at least one of a processor 1310, a memory 1330, an input interface device 1350, an output interface device 1360, and a storage device 1340, which communicate with one another through a bus 1370 so as to calculate a CDI. Also, the computing device 1300 may include a communication device 1320 coupled to a network.

The processor 1310 may include at least one CPU and/or at least one GPU and may be a semiconductor device which executes an instruction stored in the memory 1330 or the storage device 1340.

In a case where each of the elements 160, 170, 180, and 190 illustrated in FIG. 1 is implemented as a software module, the at least one CPU and/or the at least one GPU may read a corresponding software model from a storage medium, execute the read software module, and may appropriately process intermediate data and/or result data processed by the executed software module.

The memory 1330 and the storage device 1340 may include a volatile or non-volatile storage medium of various types. For example, the memory 1330 may include read only memory (ROM) and random access memory (RAM).

The communication device 1320 may be a communication module which supports wired and/or wireless communication. When the storages 110 to 150 illustrated in FIG. 1 are disposed at remote positions, the communication device 1320 may receive necessary pieces of data (for example, medical data, vital data based on a vital signal, a prediction model, standard clinic guideline data, and medical knowledge information) from the storages 110 to 150 illustrated in FIG. 1.

The storage device 1340 may include the storages 110 to 150 illustrated in FIG. 1.

The input interface device 1350 and the output interface device 1360 may each be implemented as a display unit having a touch function.

According to the embodiments of the present invention, a CDI representing a disease risk level may be calculated by using EMR/PHR data based on data associated with a standard clinic guideline or a disease screening tool and data usable as a medical/clinical basis such as a vital signal measured by a healthcare device, and thus, a disease of a user or a patient may be scientifically and objectively predicted and an optimal medical treatment may be provided based on a result of the prediction.

It will be apparent to those skilled in the art that various modifications and variations can be made in the present invention without departing from the spirit or scope of the inventions. Thus, it is intended that the present invention covers the modifications and variations of this invention provided they come within the scope of the appended claims and their equivalents.

What is claimed is:

1. A method of calculating a comprehensive disease index (CDI) by using a processor included in a computing device, the method comprising:
analyzing pieces of medical data to calculate a disease risk value;
analyzing pieces of vital data and vital data mapped to standard clinic guideline data among the pieces of vital data to calculate a disease severity value; and
analyzing the disease risk value, the disease severity value, and medical knowledge information obtained from a medical knowledge base to calculate the CDI.

2. The method of claim 1, wherein the calculating of the disease risk value comprises analyzing the medical data on the basis of a logistic regression analysis technique to calculate the disease risk value.

3. The method of claim 1, wherein the medical data comprises medical examination data, electronic medical record data, and personal health record data.

4. The method of claim 1, wherein the calculating of the disease severity value comprises:
analyzing the vital data on the basis of a machine learning model and a deep learning model to calculate a prediction probability value representing a possibility of the disease; and
analyzing the prediction probability value and the vital data mapped to the standard clinic guideline data to calculate the disease severity value.

5. The method of claim 1, wherein the calculating of the disease severity value comprises:
analyzing the vital data on the basis of a machine learning model and a deep learning model to calculate a prediction probability value representing a possibility of the disease;
converting the vital data, mapped to the standard clinic guideline data, into a scale value representing a disease severity value on the basis of a rating scale defined in a standard clinic guideline item; and
summating the prediction probability value and the scale vale to calculate the disease severity value.

6. The method of claim 1, wherein the vital data mapped to the standard clinic guideline data comprises data associated with eye tracker mapped to a standard clinic guideline item including best gaze and visual field, gyro data and electromyogram (EMG) data mapped to a standard clinic guideline item including upper extremity exercise, lower extremity exercise, and limb ataxia, and voice recognition data mapped to a standard clinic guideline item including language aphasia and dysarthria.

7. The method of claim 1, wherein the calculating of the disease severity value comprises mapping the standard clinic guideline data to the vital data on the basis of a mapping function which sets a mapping relationship between a standard clinic guideline item and the vital data.

8. The method of claim 1, wherein the calculating of the CDI comprises analyzing a correlation between the disease risk value, the disease severity value, and the medical knowledge information on the basis of Bayesian theory to calculate the CDI.

9. The method of claim 1, wherein the calculating of the CDI comprises:
calculating a posterior probability of the standard clinic guideline data when the disease risk value, the disease severity value, and the medical knowledge information are given, on the basis of Bayesian theory; and
calculating the calculated posterior probability as the CDI.

10. An apparatus for calculating a comprehensive disease index (CDI), the apparatus comprising:
a disease risk level calculation module configured to analyze pieces of medical data to calculate a disease risk value;
a disease incidence prediction module configured to analyze pieces of vital data to calculate a prediction probability value representing a possibility of the disease;
a disease severity calculation module configured to analyze vital data mapped to standard clinic guideline data among the pieces of vital data and the prediction probability value to calculate a disease severity value; and
a CDI calculation module configured to analyze the disease risk value, the disease severity value, and medical knowledge information obtained from a medical knowledge base to calculate a CDI.

11. The apparatus of claim 10, wherein the disease risk level calculation module analyzes the medical data on the basis of a logistic regression analysis technique to calculate a disease risk factor and the disease risk value corresponding to the disease risk factor.

12. The apparatus of claim 10, wherein the disease incidence prediction module analyzes each of the pieces of vital data on the basis of a machine learning model and a deep learning model to calculate a prediction probability value representing a possibility of the disease.

13. The apparatus of claim 10, wherein the disease severity calculation module comprises:
a data combiner configured to combine the standard clinic guideline data with the vital data;
a weight calculator configured to calculate a weight corresponding to the prediction probability value and a scale value converted from the vital data mapped to the standard clinic guideline data; and
an adder configured to summate the scale value, to which the weight is applied, and the prediction probability value, to which the weight is applied, to calculate the disease severity value.

14. The apparatus of claim 13, wherein the data combiner combines the standard clinic guideline data with the vital data on the basis of a mapping function which sets a mapping relationship between a standard clinic guideline item and the vital data.

15. The apparatus of claim 10, wherein the CDI calculation module calculates a posterior probability of the standard clinic guideline data when the disease risk value, the disease severity value, and the medical knowledge information are given, on the basis of a Bayesian learning model and calculates the calculated posterior probability as the CDI.
